(19) United States
(12) Patent Application Publication
(10) Pub. No.: US 2022/0076831 A1
(43) Pub. Date: Mar. 10, 2022

(54) SYSTEM AND METHOD FOR TREATMENT OPTIMIZATION USING A SIMILARITY-BASED POLICY FUNCTION

(71) Applicant: KONINKLIJKE PHILIPS N.V., Eindhoven (NL)

(72) Inventor: Yale Chang, Lincoln, MA (US)

(21) Appl. No.: 17/343,818

(22) Filed: Jun. 10, 2021

Related U.S. Application Data
(60) Provisional application No. 63/076,016, filed on Sep. 9, 2020.

(51) Int. Cl. G16H 50/20

(52) U.S. Cl. CPC G16H 50/20

(57) ABSTRACT

A method for generating an intervention recommendation by a clinical decision support system, comprising: (i) receiving a dataset of historical patient variables for a plurality of patients; (ii) training an association model with the dataset, comprising parameterizing a policy function using K-nearest neighbors and mapping physiological states and interventions to outcomes using a Q-function critic, wherein a favorable outcome is identified as a reward; (iii) receiving a physiological state for a subject; (iv) identifying K-nearest neighbors within the dataset of historical patient variables, wherein the identification is based on similarity to the physiological states of the K-nearest neighbors; (v) identifying one or more optimal interventions from among the identified K-nearest neighbors based on a highest reward for the one or more optimal interventions; (vi) generating a report comprising a recommendation for the one or more optimal interventions.

### FIELD OF THE DISCLOSURE

The present disclosure is directed generally to methods and systems for generating an optimal recommendation for treatment using a trained model.

### BACKGROUND

Clinical decision support systems are designed to provide physicians and other healthcare professionals with assistance in clinical decision-making. These support systems provide numerous advantages such as enhancing or supporting the knowledge base of the healthcare professional. Another advantage is the ability of these systems to look for patterns in historical clinical data that healthcare professionals might not be able to discern or remember due to the enormous volume of historical clinical data analyzed. A clinical decision support system can thus provide recommendations to healthcare professionals that enhance their decision-making ability.

One of the many challenges of clinical decision support systems is the design of the model utilized to identify patterns in historical clinical data, and the ability of the system to map a query patient to a pattern or a historical patient(s) in the historical clinical data for a best fit that makes the mapping sufficiently predictive of the query patient's state, best treatment, and/or outcome. Current clinical decision support systems and clinical decision support models often do not provide the best recommendations due to these limitations.

### SUMMARY OF THE DISCLOSURE

There is a continued need for clinical decision support systems and models that provide recommendations for the most optimal intervention based on historical data.

Given a query patient, it would be ideal if a clinical decision support system could: 1) suggest an optimal treatment to the query patient; and 2) support this suggestion based on similar patients in the retrospective patient database. Treatment optimization can be naturally formulated as a reinforcement learning (RL) problem. In RL, each training sample consists of a tuple (sₜ, aₜ, sₜ₊₁, rₜ₊₁), where sₜ is the patient state at the current time point (time t), aₜ is the action (intervention, treatment) applied to the patient at time t, sₜ₊₁ is the patient state at the next time point, rₜ₊₁ is the reward received at the next time point. The dynamics of patient state transition can therefore be expressed as:

p(sₜ₊₁|sₜ,aₜ) (Eq. 1)

The policy function π(aₜ|sₜ) maps the patient state to the action, which can be either deterministic or stochastic. Given a dataset containing multiple such tuples from multiple patients, which could potentially be collected from multiple hospitals, the learning objective is to maximize the state-value function v_{π}(s) w.r.t. the policy function π for all states s∈S:

$${π}^{*} = {argmax}_{π} {v}_{π} ( s ) ∀ s ∈ 𝒮 \qquad ( Eq . 2 )$$

where the state-value function v_{π}(s) is defined as the cumulative discounted rewards starting from state s as follows:

v_{π}(s)= [symbol] _{A}_{_{t:T}}_{,S}_{_{t+1:T}}_{,R}_{_{t+1:T}}[Σₖ₌₀^{T-1-t}γ^{k}Rₜ₊₁₊ₖ|Sₜ=s;π] (Eq. 3)

Most existing RL algorithms are on-line, meaning the agent is assumed to be able to apply the latest policy to collect new experience from the environment, which can be used to evaluate and improve the current policy. However, in medical applications, there is only access to a retrospective patient database. It could be costly and even unsafe to apply the policy to collect new data. Therefore, a policy must be learned from the fixed patient cohort in an offline manner. This setup is defined as batch reinforcement learning (batch RL).

Applying existing online off-policy RL algorithms to batch RL would lead to arbitrary high extrapolation error when estimating the value function. This is because the state-action pairs under consideration might not appear in the training data. To tackle this challenge, the policy function needs to be constrained so that the state-action pairs can lie within the distribution of state-action pairs in the training data.

Although existing batch RL approaches can reduce extrapolation error by constraining the policy function, the policy function is still modelled by a neural network and therefore difficult and often impossible to interpret.

Accordingly, the present disclosure is directed at inventive methods and systems for improving recommendations provided by a clinical decision support system. Various embodiments and implementations herein are directed to a clinical decision support system or method that utilizes a dataset of historical patient variables for a plurality of patients, including for each patient: (i) a physiological state over time; (ii) an intervention; (iii) an outcome of the intervention, where the outcome comprises the utility of the intervention. The system uses the historical dataset to train an association model, where the training comprises parameterizing a policy function using K-nearest neighbors and mapping physiological states and interventions to outcomes using a Q-function critic, where a favorable outcome is identified as a reward. To generate a recommendation, the system receives a physiological state for a subject and identifies K-nearest neighbors within the dataset of historical patient variables for the plurality of patients. The clinical decision support system then identifies one or more K-top ranking historical patient variables for the plurality of patients, wherein the identification is based on similarity of the physiological state of the subject to the physiological states of the K-nearest neighbors. One or more optimal interventions from among the K-top ranking historical patient variables are identified based on a highest reward for the one or more optimal interventions. To provide the recommendation, the system generates a report that includes the one or more optimal interventions.

Accordingly, the clinical decision support system parameterizes a policy function by the empirical distribution of actions applied to the soft K-nearest neighbors of the query patient. This design can naturally constrain the state-action pair within the training data distribution. Furthermore, by selecting the action corresponding to the maximal reward within those nearest neighbors, the suggested action is supported by case-based reasoning.

Generally, in one aspect, a method for generating an intervention recommendation by a clinical decision support system is provided. The method includes: (i) receiving, by the clinical decision support system, a dataset of historical patient variables for a plurality of patients, wherein the patient variables comprise for each of the plurality of patients: a physiological state over time; an intervention; an outcome of the intervention, wherein the outcome comprises the utility of the intervention; (ii) training an association model of the clinical decision support system with the dataset of historical patient variables for a plurality of patients, comprising parameterizing a policy function using K-nearest neighbors and mapping physiological states and interventions to outcomes using a Q-function critic, wherein a favorable outcome is identified as a reward; (iii) receiving, by the clinical decision support system, a physiological state for a subject; (iv) identifying, by the trained association model, K-nearest neighbors within the dataset of historical patient variables for the plurality of patients, wherein the identification is based on similarity of the physiological state of the subject to the physiological states of the K-nearest neighbors; (v) identifying, by the clinical decision support system, one or more optimal interventions from among the identified K-nearest neighbors based on a highest reward for the one or more optimal interventions; (vi) generating, by the clinical decision support system, a report comprising a recommendation for the one or more optimal interventions; and (vii) providing the report via a user interface.

According to an embodiment, the report further comprises information about the respective outcomes associated with the identified one or more optimal interventions. According to an embodiment, the report further comprises information about a similarity or distance between the subject and the one or more identified K-nearest neighbors.

According to an embodiment, the received physiological state for the subject comprises a diagnosis.

According to an embodiment, the identified one or more optimal interventions comprise a plurality of interventions, and further wherein the plurality of interventions are ranked in the generated report.

According to an embodiment, the generated report is displayed on a patient monitor.

According to an embodiment, the method further includes the steps of receiving new information about the physiological state for the subject, and updating the report based on the received new information.

According to an embodiment, a distance between the subject and a K-nearest neighbor is at least partially dependent upon a user-determined threshold.

According to another aspect, a clinical decision support system configured to generate an intervention recommendation for a subject is provided. The system includes: (i) a dataset of historical patient variables for a plurality of patients, wherein the patient variables comprise for each of the plurality of patients: a physiological state over time; an intervention; an outcome of the intervention, wherein the outcome comprises the utility of the intervention; (ii) a trained association model; (iii) a physiological state for a subject; (iv) a processor configured to: (1) train an association model with the dataset of historical patient variables for a plurality of patients, comprising parameterizing a policy function using K-nearest neighbors and mapping physiological states and interventions to outcomes using a Q-function critic, wherein a favorable outcome is identified as a reward, to generate the trained association model; (2) identify, by the trained association model, K-nearest neighbors within the dataset of historical patient variables for the plurality of patients, wherein the identification is based on similarity of the physiological state of the subject to the physiological states of the K-nearest neighbors; (3) identify one or more optimal interventions from among the identified K-nearest neighbors based on a highest reward for the one or more optimal interventions; and (4) generate a report comprising a recommendation for the one or more optimal interventions; and (v) a user interface configured to provide the report.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for generating a recommendation by a clinical decision support system, in accordance with an embodiment.

FIG. 2 is a schematic representation of a clinical decision support system, in accordance with an embodiment.

### DETAILED DESCRIPTION OF EMBODIMENTS

The present disclosure describes various embodiments of a system and method for generating an intervention recommendation. Applicant has recognized and appreciated that it would be beneficial to provide a clinical decision support system and method that provides improved intervention recommendations by identifying similar patients, interventions, and favorable outcomes. Accordingly, a clinical decision support system comprises a dataset of historical patient variables for a plurality of patients, including for each patient: (i) a physiological state over time; (ii) an intervention; (iii) an outcome of the intervention, where the outcome comprises the utility of the intervention. The system uses the historical dataset to train an association model, where the training comprises parameterizing a policy function using K-nearest neighbors and mapping physiological states and interventions to outcomes using a Q-function critic, where a favorable outcome is identified as a reward. To generate a recommendation, the system receives a physiological state for a subject and identifies K-nearest neighbors within the dataset of historical patient variables for the plurality of patients. The clinical decision support system then identifies one or more K-top ranking historical patient variables for the plurality of patients, wherein the identification is based on similarity of the physiological state of the subject to the physiological states of the K-nearest neighbors. One or more optimal interventions from among the K-top ranking historical patient variables are identified based on a highest reward for the one or more optimal interventions. To provide the recommendation, the system generates a report that includes the one or more optimal interventions.

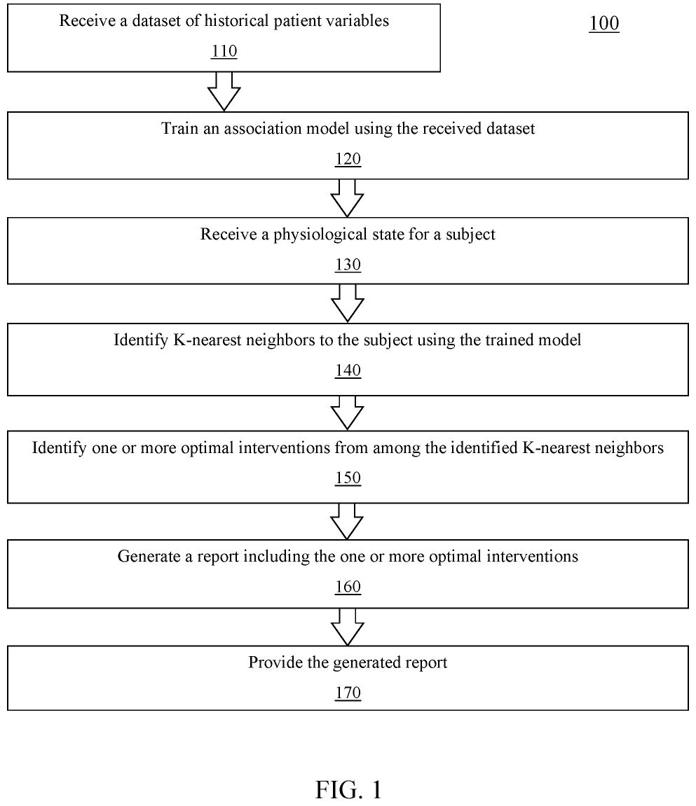

Referring to FIG. 1, in one embodiment, is a flowchart of a method 100 for generating a recommendation by a clinical decision support system. The clinical decision support system can be any of the systems described or otherwise envisioned herein.

At step 110 of the method, the clinical decision support system receives a dataset of historical patient variables for a plurality of patients. The patient variables include at least, for each of the plurality of patients: (i) a physiological state over time; (ii) an intervention; (iii) an outcome of the intervention. Further, the outcome comprises information about the utility, success, and/or lack thereof of the intervention.

According to an embodiment, the patients can be any patient or other individual that has been observed and treated. For example, a patient may be a patient in a healthcare setting such as a healthcare provider's office, an emergency setting, an in-patient facility, an out-patient facility, and/or any other setting where observations and treatments can be made.

According to an embodiment, the physiological state over time can be any feature for the patient that is relevant to medical or health or mental observation and/or treatment. For example, a feature can comprise medically relevant information about a subjection, including but not limited to demographics, physiological measurements such as vital data, injury information, physical observations, clinical test results, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types, categories, or variations of features are possible.

According to an embodiment, an intervention can be any intervention such as a treatment, modification of a treatment, medicinal or therapeutic treatment, and more. According to an embodiment, an outcome can be a change in a physiological state that potentially results from the intervention. The outcome may therefore be the status of any physiological state.

The dataset of historical patient variables for a plurality of patients can be obtained in a wide variety of different ways. The dataset may be retrieved from an electronic health database in response to a query from the system, or the electronic health database may feed the dataset to the system in response to direction to do so. Thus, the system can be in wired and/or wireless communication with an electronic health database, or the system may comprise or be a component of a system including an electronic health database or any other database comprising the dataset.

At step 120 of the method, the clinical decision support system trains an association model using the received dataset of historical patient variables for a plurality of patients, which will be the classifier utilized to provide a clinical recommendation for a subject. The classifier is trained using features extracted from the dataset of historical patient variables. According to an embodiment, training comprises parameterizing a policy function using K-nearest neighbors and mapping physiological states and interventions to outcomes using a Q-function critic, wherein a favorable outcome is identified as a reward. K can be any number, which can be determined in whole or in part by one or more of the model, the contents historical dataset, user settings, and other factors.

Policy Function Design

According to an embodiment, a policy function is designed as an element of model training. For example, one can assume that the dataset of historical patient variables [symbol] consists of N tuples, where each tuple is represented by (sₙ, aₙ, sₙ′, rₙ), where sₙ′, rₙ are the state and reward at the next time point of sₙ, aₙ. The system can parameterize the policy function using K-nearest neighbors as follows:

π(a|s;W)=Σₙ₌₁^{N}k(w^{T}sₙ,W^{T}s)δ(a−aₙ) (Eq. 4)

where k(W^{T}sₙ,W^{T}s) represents the similarity between the state of the query patient and the state of the n-th tuple. According to an embodiment, to guarantee π(a|s; W) is a valid distribution, k(W^{T}sₙ,W^{T}s) should satisfy the normalization constraint Σₙ₌₁^{N}k(W^{T}sₙ,W^{T}s)=1.

This parameterization can naturally enforce the constraint that the state-action pairs fall within the training data distribution. Furthermore, the recommended action can be supported by the actions and rewards applied to the similar patients.

Model Training

Pursuant to actor-critic models, the model can be trained as follows. First, the system can initialize Q-function (critic function) Q_{θ} and policy function (actor function) π_{W}. Next, the system can initialize target critic function Q_{θ′} and target actor function π_{W′}. In each iteration j=1, . . . , J, the system can sample a batch of transition tuples (s, a, s′, r) from the training data set.

To update the Q-function parameters, the system can apply a target actor function π_{W′} to take an action ã˜π_{W′}(s), and can apply a target critic function Q_{θ′} to define the regression target as follows:

y←r+γQ_{θ′}(s′,ã) (Eq. 5)

The system can then update Q-function parameters by stochastic gradient descent as follows:

θ←SGD(Σ(y−Q_{θ}(s,a))²) (Eq. 6)

To update the policy function parameters, the system can sample M actions from the policy network {aₘ˜π_{W}(s)}ₘ₌₁^{M}, and can update policy function parameters by stochastic gradient descent as follows:

W←SGD(− [symbol] ₐ_{_{1:M}}[(Q_{θ}(s,a)]) (Eq. 7)

To update the target function parameters, the system can proceed as follows:

θ′←τθ+(1−τ)θ′ (Eq. 8)

W′←τW+(1−τ)W′ (Eq. 9)

Compared to prior art approaches, one of the key differences of the approach described above is to parameterize the policy function using nearest neighbors. The Q-function can still be parameterized by a neural network.

According to an embodiment, the Q-function Q(s,a) approximates the expected cumulative discounted return of starting from state s, taking the action a and thereafter following policy π:

Q_{π}(s,a)= [symbol] _{A}_{_{t+1:T}}_{,S}_{_{t+1:T}}_{,R}_{_{t+1:T}}[Σₖ₌₀^{T-1-t}γ^{k}Rₜ₊₁₊ₖ|Sₜ=s,Aₜ=a;π] (Eq. 10)

According to an embodiment, the target critic function and target actor functions are introduced to define the regression target following the Bellman equation. According to an embodiment, γ is the discounting factor when computing the cumulative discounted rewards. According to an embodiment, τ is the contribution from the Q-function parameters when updating the parameters of the target Q-function.

Following step 120, the clinical decision support system comprises a trained classifier that can be utilized to provide a treatment recommendation for a subject as described or otherwise envisioned herein. The trained classifier can be static such that it is trained once and is utilized for classifying. According to another embodiment, the trained classifier can be more dynamic such that it is updated or re-trained using subsequently available training data. The updating or re-training can be constant or can be periodic.

At step 130 of the method, the clinical decision support system receives a physiological state for a subject. According to an embodiment, the subject can be any patient or other individual that is being observed and/or treated. For example, the subject may be a patient in a healthcare setting such as a healthcare provider's office, an emergency setting, an in-patient facility, an out-patient facility, and/or any other setting where observations and treatments can be made.

According to an embodiment, the physiological state can be any feature for the patient that is relevant to medical or health or mental observation and/or treatment. For example, a feature can comprise medically relevant information about a subjection, including but not limited to demographics, physiological measurements such as vital data, injury information, physical observations, clinical test results, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types, categories, or variations of features are possible.

The physiological state for the subject can be provided to the clinical decision support system in a wide variety of ways. For example, the physiological state can be manually input into the clinical decision support system. The physiological state may be retrieved from an electronic health database in response to a query from the system, or the electronic health database may feed the physiological state to the system in response to direction to do so. Thus, the system can be in wired and/or wireless communication with an electronic health database, or the system may comprise or be a component of a system including an electronic health database or any other database comprising the physiological state.

According to an embodiment, after training the model, given a query patient represented by the state vector s, the learned policy function can be applied to compute the K nearest neighbors of s in the low dimensional space. This is achieved by ranking the following kernel function value in descending order as follows:

(k(W^{T}s₁,W^{T}s), . . . ,(W^{T}sₙ,W^{T}s), . . . ,(W^{T}s_{N},W^{T}s)) (Eq. 11)

The system can denote the K-top ranking samples as {(sᵢ_{₁},aᵢ_{₁}), . . . , (sᵢ_{ₖ},aᵢ_{ₖ}), . . . , (sᵢ_{_{K}}, aᵢ_{_{K}})}, for each distinct action, and its expected reward can be evaluated as follows:

$$q ( a ) = \frac{{\sum }_{k = 1}^{K} 𝕀 [ {a}_{{i}_{k}} = a ] {Q}_{θ} ( {s}_{{i}_{k}} , {a}_{{i}_{k}} )}{{\sum }_{k = 1}^{K} 𝕀 [ {a}_{{i}_{k}} = a ]} \qquad ( Eq . 12 )$$

According to an embodiment, the optimal action would be the one corresponding to the maximal expected reward as follows:

$${a}^{*} = {argmax}_{a} q ( a ) \qquad ( Eq . 13 )$$

Therefore, at step 140 of the method, the clinical decision support system identifies, by the trained association model, K-nearest neighbors within the dataset of historical patient variables for the plurality of patients. As just one example, as described above, given a query patient represented by the state vector s the learned policy function can be applied to compute the K nearest neighbors of s in the low dimensional space. For example, this can be achieved by ranking the following kernel function value in descending order as follows:

(k(W^{T}s₁,W^{T}s), . . . ,(W^{T}sₙ,W^{T}s), . . . ,(W^{T}s_{N},W^{T}s)) (Eq. 11)

According to an embodiment, a user may set one or more thresholds or parameters which are utilized to determine an allowable and/or preferred distance to a nearest neighbor. If the distance to a neighbor exceeds the user-set threshold or parameter, the classifier will identify the neighbor as being too different from the subject and thus not suitable for providing a recommendation. If no neighbors are detected within the allowable and/or preferred distance, based on the user-set threshold or parameter, the clinical decision support system may not be able to generate a recommendation. Rather than providing a report, the system may provide an indication that no recommendation could be generated. According to an embodiment, the system may provide a prompt to enable or allow a larger distance to neighbors to allow for the generation of a recommendation using those more distant neighbors.

Thus, following step 140 of the method, the system comprises K-nearest neighbors of the subject from the dataset.

At step 150 of the method, the clinical decision support system identifies one or more optimal interventions from among the identified K-nearest neighbors. According to an embodiment, the selection is based at least in part on a highest reward for the one or more optimal interventions. As just one example, as described above, the system can denote the K-top ranking samples as {(sᵢ_{₁},aᵢ_{₁}), . . . , (sᵢ_{ₖ}, aᵢ_{ₖ}), . . . , sᵢ_{_{K}},aᵢ_{_{K}})}, for each distinct action, and its expected reward can be evaluated as follows:

$$q ( a ) = \frac{{\sum }_{k = 1}^{K} 𝕀 [ {a}_{{i}_{k}} = a ] {Q}_{θ} ( {s}_{{i}_{k}} , {a}_{{i}_{k}} )}{{\sum }_{k = 1}^{K} 𝕀 [ {a}_{{i}_{k}} = a ]} \qquad ( Eq . 12 )$$

According to an embodiment, the optimal action would be the one corresponding to the maximal expected reward as follows:

$${a}^{*} = {argmax}_{a} q ( a ) \qquad ( Eq . 13 )$$

Thus, following step 150 of the method, the system comprises one or more optimal interventions from among the identified K-nearest neighbors.

At step 160 of the method, the clinical decision support system generates a report that includes the identified one or more optimal interventions. The optimal intervention can be any intervention taken for the identified K-nearest neighbor(s), such as a treatment, modification of a treatment, medicinal or therapeutic treatment, and more. According to an embodiment, the report may also comprise information about the outcome of the intervention among the one or more identified K-nearest neighbor(s). The report may also comprise information about the similarity between the subject and the one or more identified K-nearest neighbor(s). The report may further comprise anything that may be helpful to the clinician in their decision-making process. The report can be generated by the clinical decision support system using any method for gathering, processing, and/or collating the reported information.

According to an embodiment, the identified one or more optimal interventions and comprise a plurality of interventions, which may be identical, similar, or different. The plurality of interventions can be ranked in the generated report. The ranking may be determined in whole or in part by a distance or other similarity metric between the subject and the K-nearest neighbors in the historical dataset from which the plurality of interventions were identified. For example, the top-ranking optimal intervention may be the intervention identified for the closest K-nearest neighbor to the subject.

At step 170 of the method, the report is provided via a user interface or other communication method. The user interface can be any device or system that allows information to be conveyed and/or received, and may include a display, a mouse, and/or a keyboard for receiving user commands. The user interface may be located with one or more other components of the system, or may located remote from the system and in communication via a wired and/or wireless communications network. For example, the report may be displayed on a screen, printed, texted, emailed, displayed via a wearable device, or provided using any other method for communicating information. According to an embodiment, the report is provided to a clinician, which enables the clinician to evaluate the recommendation.

According to an embodiment, the clinical decision support system can be configured to update the optimal recommendation. The update may be based on a predetermined or user-set time period, updated information about the subject such as new or updated information about a physiological state of the subject, or for other reasons. For example, the system may be in communication with a database or other source of medical information about the subject, and may be triggered to provide a recommendation or updated recommendation upon receipt of new information. Accordingly, the clinical decision support system can be configured to generate and provide optimal intervention recommendations in real-time. The real-time optimal intervention recommendation(s) can be displayed on a screen such as a patient monitor or other display, and can be updated in real-time as the recommendation changes.

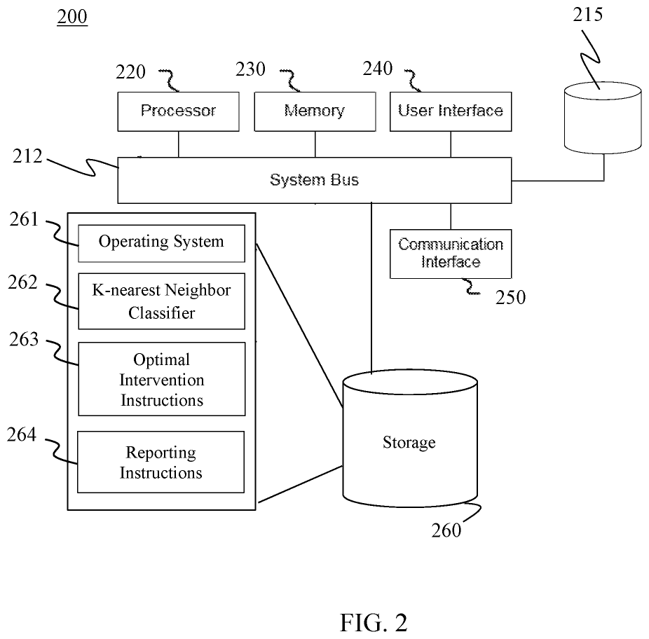

Referring to FIG. 2, in one embodiment, is a schematic representation of a clinical decision support system 200. System 200 may be any of the systems described or otherwise envisioned herein, and may comprise any of the components described or otherwise envisioned herein.

According to an embodiment, system 200 comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 620 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may comprise or be in remote or local communication with a database or data source 215. Database 215 may be a single database or data source or multiple. Database 215 may comprise the input data which may be used to train the system, as described and/or envisioned herein, such as the dataset of historical patient variables for a plurality of patients. This dataset may include, for example, for at least some of the patients: (i) a physiological state over time; (ii) an intervention; (iii) an outcome of the intervention, where the outcome comprises the utility of the intervention.

According to an embodiment, storage 260 of system 200 may store one or more algorithms and/or instructions to carry out one or more functions or steps of the methods described or otherwise envisioned herein. For example, processor 220 may comprise one or more of a trained classifier 262, optimal intervention instructions 263, and reporting instructions 264.

According to an embodiment, the trained classifier 262 is configured to identify, from a dataset, K-nearest neighbors to a subject. The classifier is trained using features extracted from the dataset of historical patient variables. According to an embodiment, training comprises parameterizing a policy function using K-nearest neighbors and mapping physiological states and interventions to outcomes using a Q-function critic, wherein a favorable outcome is identified as a reward. K can be any number, which can be determined in whole or in part by one or more of the model, the contents historical dataset, user settings, and other factors.

According to an embodiment, the optimal intervention instructions 263 direct the system to identify, from among the K-nearest neighbors, one or more optimal interventions. The optimal intervention can be any intervention taken for the identified K-nearest neighbor(s), such as a treatment, modification of a treatment, medicinal or therapeutic treatment, and more. According to an embodiment, the selection is based at least in part on a highest reward for the one or more optimal interventions.

According to an embodiment, the reporting instructions 264 direct the system to generate a report comprising the identified one or more optimal interventions. According to an embodiment, the report may also comprise information about the outcome of the intervention among the one or more identified K-nearest neighbor(s). The report may also comprise information about the similarity between the subject and the one or more identified K-nearest neighbor(s). The report may further comprise anything that may be helpful to the clinician in their decision-making process. The report may comprise any other information received or generated by the risk analysis system.

The reporting instructions 264 also direct the system to display the report on a display of the system or provide the report via any other communication mechanism or method. For example, the report may be communicated by wired and/or wireless communication to another device. For example, the system may communicate the report to a mobile phone, computer, laptop, wearable device, and/or any other device configured to allow display and/or other communication of the report.

According to an embodiment, the clinical decision support system is configured to process many thousands or millions of datapoints during the training of the classifier, identifying K-nearest neighbors within the dataset of historical patient variables for the plurality of patients, and identifying one or more optimal interventions from among the identified K-nearest neighbors, among other calculations and analyses. This can require millions or billions of calculations to generate a single report comprising the identified one or more optimal interventions, and/or any other information. Generating this information and providing the report comprises a process with a volume of calculation and analysis that a human brain cannot accomplish in a lifetime, or multiple lifetimes.

By providing such an improved recommendation for optical intervention, the clinical decision support methods and systems described or otherwise envisioned herein improve the ability of clinicians or other decisionmakers to assess a subject, provide an intervention, and improve outcomes. It also increases the decisionmaker's confidence in the underlying system. The generated and provided recommendation or call to action improves the care of the subject by providing a clearer picture of the subject's condition and a better prediction of the future. Improved interventions, such as that performed by the novel systems and methods described or otherwise envisioned herein, saves lives and saves millions of dollars a year in healthcare costs, when applied in the healthcare setting.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for generating an intervention recommendation by a clinical decision support system, comprising:
receiving, by the clinical decision support system, a dataset of historical patient variables for a plurality of patients, wherein the patient variables comprise for each of the plurality of patients: (i) a physiological state over time; (ii) an intervention; (iii) an outcome of the intervention, wherein the outcome comprises the utility of the intervention;
training an association model of the clinical decision support system with the dataset of historical patient variables for a plurality of patients, comprising parameterizing a policy function using K-nearest neighbors and mapping physiological states and interventions to outcomes using a Q-function critic, wherein a favorable outcome is identified as a reward;
receiving, by the clinical decision support system, a physiological state for a subject;
identifying, by the trained association model, K-nearest neighbors within the dataset of historical patient variables for the plurality of patients, wherein the identification is based on similarity of the physiological state of the subject to the physiological states of the K-nearest neighbors;
identifying, by the clinical decision support system, one or more optimal interventions from among the identified K-nearest neighbors based on a highest reward for the one or more optimal interventions;
generating, by the clinical decision support system, a report comprising a recommendation for the one or more optimal interventions; and
providing the report via a user interface.

2. The method of claim 1, wherein the report further comprises information about the respective outcomes associated with the identified one or more optimal interventions.

3. The method of claim 1, wherein the report further comprises information about a similarity or distance between the subject and the one or more identified K-nearest neighbors.

4. The method of claim 1, wherein the received physiological state for the subject comprises a diagnosis.

5. The method of claim 1, wherein the identified one or more optimal interventions comprise a plurality of interventions, and further wherein the plurality of interventions are ranked in the generated report.

6. The method of claim 1, wherein the generated report is displayed on a patient monitor.

7. The method of claim 1, further comprising the steps of receiving new information about the physiological state for the subject, and updating the report based on the received new information.

8. The method of claim 1, wherein a distance between the subject and a K-nearest neighbor is at least partially dependent upon a user-determined threshold.

9. A clinical decision support system configured to generate an intervention recommendation for a subject, comprising:
a dataset of historical patient variables for a plurality of patients, wherein the patient variables comprise for each of the plurality of patients: (i) a physiological state over time; (ii) an intervention; (iii) an outcome of the intervention, wherein the outcome comprises the utility of the intervention;
a trained association model;
a physiological state for a subject;
a processor configured to: (i) train an association model with the dataset of historical patient variables for a plurality of patients, comprising parameterizing a policy function using K-nearest neighbors and mapping physiological states and interventions to outcomes using a Q-function critic, wherein a favorable outcome is identified as a reward, to generate the trained association model; (ii) identify, by the trained association model, K-nearest neighbors within the dataset of historical patient variables for the plurality of patients, wherein the identification is based on similarity of the physiological state of the subject to the physiological states of the K-nearest neighbors; (iii) identify one or more optimal interventions from among the identified K-nearest neighbors based on a highest reward for the one or more optimal interventions; and (iv) generate a report comprising a recommendation for the one or more optimal interventions; and
a user interface configured to provide the report.

10. The system of claim 9, wherein the report further comprises information about the respective outcomes associated with the identified one or more optimal interventions.

11. The system of claim 9, wherein the report further comprises information about a similarity or distance between the subject and the one or more identified K-nearest neighbors.

12. The system of claim 9, wherein the physiological state for the subject comprises a diagnosis.

13. The system of claim 9, wherein the identified one or more optimal interventions comprise a plurality of interventions, and further wherein the plurality of interventions are ranked in the generated report.

14. The system of claim 9, wherein the generated report is displayed on a patient monitor.

15. The system of claim 9, wherein a distance between the subject and a K-nearest neighbor is at least partially dependent upon a user-determined threshold.
